(19) United States
(12) Patent Application Publication
(10) Pub. No.: US 2026/0364095 A1
(43) Pub. Date: Nov. 19, 2026

(54) TREATMENT OF PSORIASIS

(71) Applicant: TRADOVA (PTY) LTD, Johannesburg (ZA)

(72) Inventor: Rafeek HASSEN, Durban (ZA)

(21) Appl. No.: 19/490,109

(22) PCT Filed: May 31, 2024
(86) PCT No.: PCT/IB2024/055328

§ 371 Date: Dec. 4, 2025

(30) Foreign Application Priority Data
Jun. 6, 2023 (ZA) 2023/05965

(51) Int. Cl. A61K 31/58; A61K 9/00; A61K 9/06; A61K 31/60; A61K 47/44; A61K 47/46; A61P 17/06

(52) U.S. Cl. CPC A61K 31/58; A61K 9/0014; A61K 9/06; A61K 31/60; A61K 47/44; A61K 47/46; A61P 17/06

(57) ABSTRACT

This invention relates to a topical formulation comprising as active ingredients, one or more Class 6 Low Potency corticosteroid(s), an emulsifying ointment BP, honey, one or more natural oil(s), and optionally salicylic acid, to a method of producing such topical formulation, and to a method of treatment of psoriasis or the symptoms thereof in a subject with the application of the topical formulation of the invention to a subject in need thereof.

### FIELD OF THE INVENTION

This invention relates to the treatment of psoriasis.

### BACKGROUND TO THE INVENTION

Psoriasis is an inflammatory condition that is characterized by many different types of symptoms, most typically a rash with itchy, scaly patches, most commonly on the knees, elbows, trunk and scalp. Psoriasis is a common, long-term, i.e. chronic, skin disease.

In addition, psoriasis symptoms can alternate between periods of flare ups, during which the symptoms are intense, and periods of remission during which the symptoms clear up. Remission periods last for an average of one to twelve months at a time. However, the duration of both flare ups and periods of remission can be challenging to predict.

Psoriasis may therefore be characterised in essentially three phases, i.e.:
an acute phase characterized by itching and breaking up of scales/rashes,
a scaly phase characterized by thickening and or discoloration of affected areas, and
a remission phase where there is no itching but mild scaling or dryness or redness of skin.

There are various existing products that have been used commercially to treat psoriasis and the symptoms thereof, some of which are included in Table 1:

TABLE 1
Existing products used to treat psoriasis and the symptoms thereof

| Product | Ingredients |
|---|---|
| Cosalic topical formula | Salicylic Acid and Coal Tar |
| Triderma psoriasis | Salicylic acid, Urea, Oat |
| control | protein, Vit B & E |
| Cera VE psoriasis | Salicylic acid, Lactic Acid, |
| cleanser | Alpha Hydroxy Acid |
| Mg 217 psoriasis ointment | Silver crumbs containing |
| intensive strength | Coal Tar 113.4 g |
| Sonatur natural | Rose Geranium oil, Lanolin, |
| psoriasis cream | white oil cosmetic grade |
| Linotar cream | Coal Tar, Vit E |
| H&B natural PSO | Dead Sea minerals, Vit E, |
| psoriasis cream | Aloe, Coconut & Olive Oil |
| Dovate ointment | Clobetasol Propionate |
| Dovonex ointment | Calcipotriene Vitamin D |

However, as a formulating pharmacist with more than 20 years of experience, the applicant has found that none of these formulations were effective long term, and that patients would experience, at most, temporary relief from their psoriasis symptoms for a short period, followed by a rebound back to the original severity of psoriasis experienced.

It would be useful if an alternative product could be developed that provided long term relief from psoriasis and the symptoms thereof.

### SUMMARY OF THE INVENTION

ACCORDING TO A FIRST ASPECT OF THE INVENTION THERE IS PROVIDED a topical formulation comprising or consisting of, as active ingredients, one or more Class 6 Low Potency corticosteroid(s), an emulsifying ointment BP, honey, one or more natural oil(s), and optionally salicylic acid.

In one possible embodiment of the first aspect of the invention, the topical formulation consists of, as active ingredients, one or more Class 6 Low Potency corticosteroid(s), an emulsifying ointment BP, honey, one or more natural oil(s) and optionally salicylic acid.

It is to be appreciated that Class 6 Low Potency corticosteroids are well known to those skilled in the art. However, in particular, the Class 6 Low Potency corticosteroid(s) may be selected from the group consisting of any one or more Class B, Triamcinolone Acetonide Type corticosteroid(s) or any one or more Class D, Betamethasone Dipropionate Type corticosteroid(s) or any combination thereof.

For example, the Class B, Triamcinolone Acetonide Type corticosteroid(s) may be selected from any one or more of:
Desonide 0.05% (w/w);
Fluocinolone acetonide 0.01% (w/w);
Triamcinolone acetonide 0.025% (w/w); or
Triamcinolone diacetate 0.025% (w/w), or any combination thereof.

For example, the Class D, Betamethasone Dipropionate Type corticosteroid(s) may be selected from any one or more of:
Alclometasone dipropionate 0.05% (w/w); or
Betamethasone valerate 0.1% (w/w), or any combination thereof.

In one possible embodiment of the first aspect of the invention, the Class 6 Low Potency corticosteroid is fluocinolone acetonide. In particular, the fluocinolone acetonide may have the chemical name pregna-1,4-diene-3,20-dione, 6,9-difluoro-11, 21-dihydroxy-16, 17-[(1-methylethylidene)bis(oxy)]-, (6α, 1β, 16α) and the structural formula as shown in Formula I.

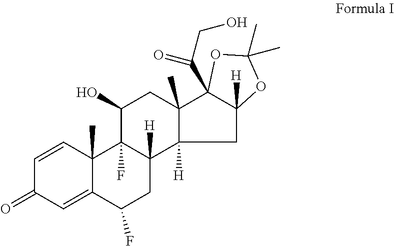

The formulation may comprise, by weight of the total formulation, from about 0.0025% to about 0.2%, including from about 0.0025% to about 0.025, of the Class 6 Low Potency corticosteroid.

In one possible embodiment of the invention, the formulation comprises, by weight of the total formulation, between about 0.00625% and 0.01% of the Class 6 Low Potency corticosteroid, including about 0.0025% of the Class 6 Low Potency corticosteroid.

In another possible embodiment of the invention, the formulation comprises, by weight of the total formulation, about 0.025% of the Class 6 Low Potency corticosteroid.

In yet another possible embodiment of the invention, the formulation comprises about 0.2% by weight of the total formulation of the Class 6 Low Potency corticosteroid.

In one possible embodiment of the invention, the Class 6 Low Potency corticosteroid is fluocinolone acetonide.

The one or more corticosteroid(s) may be formulated as a cream, a foam or an ointment. In particular, the formulation may be an ointment.

The honey is preferably raw, unprocessed honey. In this regard, reference is made to honey in the form in which it is present in a hive in which it is produced. In particular, the formulation may comprise from about 5% to about 10%, by weight of the total formulation, of the honey. For example, the formulation may comprise about 5%, or about 6%, or about 7%, or about 8%, or about 9%, or about 10% by weight of the total formulation, of the honey.

The one or more natural oil(s) is preferably olive oil. More particularly, the olive oil is virgin olive oil extracted via a cold process method. The formulation may comprise from about 5% to about 10%, by weight of the total formulation, of the one or more natural oil(s). For example, the formulation may comprise about 5%, or about 6%, or about 7%, or about 8%, or about 9%, or about 10% by weight of the total formulation, of the one or more natural oil(s).

In a particular embodiment, the emulsifying ointment BP consists of:
Emulsifying Wax—30% (w/w);
White Soft Paraffin—50% (w/w); and
Liquid Paraffin—20% (w/w).

Typically, the formulation comprises emulsifying ointment BP to make up the final formulation to 100% by weight of the total formulation.

The salicylic acid may, in particular, be that of Formula II

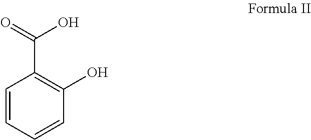

The formulation may comprise from about 2% to about 10%, by weight, of the salicylic acid. For example, the formulation may comprise about 2%, or about 3%, or about 4%, or about 5%, or about 6%, or about 7%, or about 8%, or about 9%, or about 10% by weight of the total formulation, of the salicylic acid.

ACCORDING TO A SECOND ASPECT OF THE INVENTION THERE IS PROVIDED a formulation according to the first aspect of the invention for use in the treatment of psoriasis or of at least one symptom arising from psoriasis in a subject in need thereof.

In particular, the subject is a human. The formulation may be in the form of a cream or an ointment. In particular, the formulation may be an ointment.

ACCORDING TO A THIRD ASPECT OF THE INVENTION THERE IS PROVIDED a use of the compounds selected from the group comprising or consisting of: one or more Class 6 Low Potency corticosteroid(s), an emulsifying ointment BP, honey, one or more natural oil(s), and optionally salicylic acid as described in the first aspect of the invention in the manufacture of a formulation for the treatment of psoriasis or of at least one symptom arising from psoriasis in a subject in need thereof.

In particular, the subject is a human. The formulation may be in the form of a cream or an ointment. In particular, the formulation may be an ointment.

ACCORDING TO A FOURTH ASPECT OF THE INVENTION THERE IS PROVIDED a method of treating psoriasis or at least one symptom arising from psoriasis in a subject in need thereof, the method comprising topical application of the formulation according to the first aspect of the invention to an area of skin of the subject affected by psoriasis or at least one symptom arising from psoriasis.

The method may include a first step of selecting a particular formulation according to the first aspect of the invention wherein the particular formulation contains a concentration of the one or more Class 6 Low Potency corticosteroid(s) appropriate for a particular phase of psoriasis or symptom(s) thereof of the subject. The particular phase may be selected from one of:
an acute phase, typically characterized by itching, inflammation, scales and/or rashes;
a scaly phase, typically characterized by thickening and/or discoloration of affected areas with scale formation; and
a remission phase, where there is typically no itching but mild scaling, dryness and/or inflammation of skin.

In one possible embodiment of the fourth aspect of the invention where the subject is in the acute phase, the formulation may be an acute phase formulation having a concentration of the one or more Class 6 Low Potency corticosteroid(s) of about 0.0125% by weight. Optionally, in this embodiment of the fourth aspect of the invention, salicylic acid is included in the formulation. For example, the formulation may comprise about 2%, or about 3%, or about 4%, or about 5%, or about 6%, or about 7%, or about 8%, or about 9%, or about 10% by weight of the total formulation, of the salicylic acid.

In this embodiment of the invention, the formulation may be applied twice a day on affected areas of skin of the subject until resolution of acute phase symptoms of psoriasis whereupon the subject enters the remission phase, followed by a phased discontinuation of use of the acute phase formulation comprising once-daily application for about 5 days, followed by once-daily application on alternate days for the next about 10 days. Typically, the subject may then use a formulation of the first aspect of the invention having a concentration of the one or more Class 6 Low Potency corticosteroid(s) of about 0.0025% by weight (i.e. a remission phase formulation) while in remission phase. Typically, no salicylic acid is included in the remission phase formulation.

In a further possible embodiment of the fourth aspect of the invention where the subject is in the scaly phase, the concentration of the one or more Class 6 Low Potency corticosteroid(s) in the formulation may be about 0.00625% by weight (i.e. a scaly phase formulation) and the formulation may further comprise from about 2% to about 10%, by weight, of salicylic acid. For example, the formulation may comprise about 2%, or about 3%, or about 4%, or about 5%, or about 6%, or about 7%, or about 8%, or about 9%, or about 10% by weight of the total formulation, of the salicylic acid.

In this embodiment of the invention, the scaly phase formulation may be applied twice a day on affected areas of skin of the subject until resolution of scaly phase symptoms of psoriasis, whereupon the subject enters the remission phase, followed by a phased discontinuation of use of the scaly phase formulation comprising once daily application for about 5 days, followed by once-daily application on alternate days for the next about 10 days. Typically, the subject may then use a formulation of the invention having a concentration of the one or more Class 6 Low Potency corticosteroid(s) in of about 0.0025% by weight (i.e. a remission phase formulation) while in remission phase. Typically, no salicylic acid is included in the remission phase formulation.

In yet a further possible embodiment of the fourth aspect of the invention where the subject is in the remission phase, the concentration of the one or more Class 6 Low Potency corticosteroid(s) in the formulation may be about 0.0025% by weight (i.e. the remission formulation). Typically, no salicylic acid is included in the remission phase formulation.

ACCORDING TO A FIFTH ASPECT OF THE INVENTION THERE IS PROVIDED a method of producing a formulation according to the first aspect of the invention, the method including mixing one or more Class 6 Low Potency corticosteroid(s), an emulsifying ointment BP, honey, one or more natural oil(s), and optionally salicylic acid as described in the first aspect of the invention.

### EXAMPLES

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

### Comparison of Formulations of the Invention with Existing Commercial Products

Observations from five anecdotal case studies are set out below, comparing the efficacy of the topical formulations of the present invention with existing commercial products that had been prescribed by the patient's physicians. Depending on the phase of the condition, different formulations of the present invention were administered, i.e.:
P1 for Acute Phase.
P2 for Scaly Phase.
P3 for Remission Phase

### Treatment Formulations

### P1 for Acute Phase Treatment

Formula for 100 g
Class 6 Low Potency corticosteroid such as fluocinolone acetonide—0.0125% (w/w)
Raw Honey—5% (w/w)
Olive Oil—5% (w/w)
Emulsifying ointment BP to 100 g (w/w)
Optionally salicylic acid—5% (w/w)

If being included, first weigh out the salicylic acid and pulverize it to a powder. Next add the Class 6 Low Potency corticosteroid 0.00625% (w/w) and mix thoroughly until combined. If no salicylic acid is to be included, first weigh out the required amount of emulsifying ointment BP, then add the Class 6 Low Potency corticosteroid and mix thoroughly until combined. Add the honey and olive oil gradually, mixing continuously until homogenous.

The resultant product is a smooth, stable, pleasantly fragrant cream that is applied twice a day only on the affected areas.

After relief has been obtained this product is discontinued over a two-week period by applying daily for five days, then alternate days for the next ten days. The subject will then be in remission and may use the remission phase treatment as set out below.

### P2 for Scaly Phase Treatment

Formula for 100 g
Class 6 Low Potency corticosteroid such as fluocinolone acetonide—0.00625% (w/w)
Raw Honey—5% (w/w)
Olive Oil—5% (w/w)
Salicylic Acid—5% (w/w)
Emulsifying ointment BP to 100 g (w/w)

First weigh out the salicylic acid and pulverize it to a powder. Next add the Class 6 Low Potency corticosteroid 0.00625% (w/w) and mix thoroughly until combined. Weigh out the required amount of emulsifying ointment BP, then add the Class 6 Low Potency corticosteroid and mix thoroughly until combined. Finally add the honey and olive oil gradually, mixing continuously until homogenous.

The resultant product is a smooth, stable, pleasantly fragrant cream that is applied twice a day only on the affected areas.

As the scaly phase is characterized by hard skin, scales, dryness and even itching, there is a need to add salicylic acid which has keratolytic affects.

After relief has been obtained this product is discontinued over a two-week period by applying daily for five days, then alternate days for the next ten days. The subject will then be in remission and may use the remission phase treatment as set out below.

### P3 for Remission Phase Treatment

Formula for 100 g
Class 6 Low Potency corticosteroid such as fluocinolone acetonide—0.0025% (w/w)
Raw Honey—10% (w/w)
Olive Oil—10% (w/w)
Emulsifying ointment BP to 100 g (w/w)

Weigh out the required amount emulsifying ointment PB and add the Class 6 Low Potency corticosteroid 0.0025% (w/w) and mix thoroughly until combined. Finally add the honey and olive oil gradually, mixing continuously until homogenous.

The resultant product is a smooth, stable, pleasant fragrant cream that is applied sparingly twice a day only on the affected areas.

Treatment with this formulation for remission phase must be affected after treatment with formulations for acute phase and scaly phase.

In the remission phase there is a desire aims to keep the skin ‘calm’ and therefore to prevent a relapse a very small amount of the Class 6 Low Potency corticosteroid of 0.0025% (w/w) was surprisingly found to be effective to prevent inflammation and remove the itching that may occur during the remission phase.

### Case Studies

### Case 1

A male adult, aged 50 years had been treated for about 1 year with a combination of commercially available medications, viz. Chlorphenoxamine hydrochloride (Allergex®) cream; Nomospore (Bacitracin, Neomycin, Polymyxin B and Cocoa Butter, Cotton Seed Oil, Olive Oil, Sodium Pyruvate, Vitamin E, White Petrolatum); and Clobetasol propionate (Dovate®). At the time of first treatment with the formulation P1 of the present invention, the subject presented with itchy, broken skin including cuts on lower leg from acute phase psoriasis.

By the end of week 1 of treatment with formulation P1, the subject reported experiencing relief of the itchiness in 2 to 3 days and the skin appearance showed less inflammation than at the start of treatment. By the end of week 2 of treatment, the cuts had started to heal and skin appearance showed less inflammation than at week 1 of treatment. By the end of week 3, the subject's skin appearance was almost normal. At the end of week 4, the subject was placed on formulation P3 for maintenance of his psoriasis and no flare-ups were noted after three months of follow-up.

### Case 2

A male adult, aged 35 years had been treated for over 3 years with a combination of commercially available medications, viz. Chlorphenoxamine hydrochloride (Allergex®) tablets and cream Vaseline® (petroleum jelly, palmitic acid, stearic acid, mineral oil, glycerin, glyceryl stearate, cetyl alcohol, and potassium hydroxide); Dovonex® cream (calcipotriene monohydrate equivalent to 50 μg/g anhydrous calcipotriene in a cream base of cetearyl alcohol, ceteth-20, diazolidinyl urea, dichlorobenzyl alcohol, dibasic sodium phosphate, edetate disodium, dl-alpha tocopherol, glycerin, mineral oil, petrolatum. At the time of first treatment with the formulation P2 of the present invention, the subject presented with very dry, scaly and itchy skin from the ankle upwards on both legs from scaly phase psoriasis.

By the end of week 1 of treatment with formulation P2, the subject reported experiencing relief of the itchiness and the skin appearance showed most scales had resolved compared to the start of treatment. By the end of week 2 of treatment, the subject was able to move to formulation P3 for maintenance of his psoriasis. At one month from the start of treatment, the subject had a slight flare-up and was treated for two days with formulation P1, which resolved the flare-up and the subject then was able to return to formulation P3 for maintenance of his psoriasis with no further flare-ups.

### Case 3

A female child subject, aged 6 years had been treated for about 2 years with Unguentum emulsificans aquosum. At the time of first treatment with the remission formulation P3 of the present invention, the subject presented with very itchy inflamed patches of skin on the body. The subject reported relief within 1 week of treatment and has remained without symptoms for 6 months with the use of formulation P3.

### Case 4

A female adult, aged 65 years had been treated for about 2 years with a combination of commercially available medications, viz. Skincalm™ (Infused oil (calendula, Oregon grape root, comfrey leaf, comfrey root, chamomile, olive oil), distilled water, shea butter, glycerin, beeswax, veg emulsifying wax, borage oil, emu oil, vitamin E, potassium sorbate); Zam-buk™ (paraffin wax, pale resin (colophony), eucalyptus oil, camphor, thyme oil, and sassafras oil). At the time of first treatment with the remission formulation P3 of the present invention, the subject presented with dry, itchy skin on both shins with discolouration from psoriasis. The subject reported relief within 1 week of treatment and has remained without symptoms for 6 months with the use of formulation P3.

### Case 5

A female child, aged 8 years had been treated for about 6 months with a combination of commercially available medications, viz. Olive oil followed by a Dovate®, coal tar and salicylic acid mixture. At the time of first treatment with the scaly phase formulation P2 of the present invention, the subject presented with dry, itchy skin on the nape of the neck and a dry and scaly scalp from psoriasis. The subject reported relief and the scales resolved within 3 days of treatment with P2. The subject was then moved onto the remission formulation P3 and has remained without symptoms for 8 months without flare-up.

1. A topical formulation consisting of, as active ingredients, one or more Class 6 Low Potency corticosteroid(s) selected from the group consisting of any one or more Class B, Triamcinolone Acetonide Type corticosteroid(s) or any one or more Class D, Betamethasone Dipropionate Type corticosteroid(s) or any combination thereof, an emulsifying ointment BP (British Pharmacopeia), raw honey, olive oil, and optionally salicylic acid.

2. The topical formulation according to claim 1, wherein the Class B, Triamcinolone Acetonide Type corticosteroid(s) are selected from any one or more of:
Desonide 0.05% (w/w);
Fluocinolone acetonide 0.01% (w/w);
Triamcinolone acetonide 0.025% (w/w); or
Triamcinolone diacetate 0.025% (w/w), or any combination thereof.

3. The topical formulation according to claim 1, wherein the Class D, Betamethasone Dipropionate Type corticosteroid(s) are selected from any one of more of:
Alclometasone dipropionate 0.05% (w/w); or
Betamethasone valerate 0.1% (w/w), or any combination thereof.

4. The topical formulation according to claim 1, wherein the Class 6 Low Potency corticosteroid is fluocinolone acetonide having the chemical name pregna-1,4-diene-3,20-dione, 6,9-difluoro-11, 21-dihydroxy-16, 17-[(1-methylethylidene)bis(oxy)]-, (6α, 1β, 16α) and the structural formula as shown in Formula I:

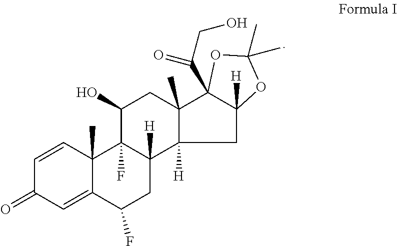

5. The topical formulation according to claim 4, wherein, by weight of the total formulation, the fluocinolone acetonide concentration is from about 0.0025% to about 0.0125.

6. The topical formulation according to claim 5, wherein, by weight of the total formulation, the fluocinolone acetonide concentration is about 0.0125%.

7. The topical formulation according to claim 5, wherein, by weight of the total formulation, the fluocinolone acetonide concentration is about 0.00625%.

8. The topical formulation according to claim 7, wherein, by weight of the total formulation, the fluocinolone acetonide concentration is about 0.0025%.

9. The topical formulation according to claim 1, wherein the formulation comprises from about 5% to about 10%, by weight of the total formulation of the honey.

10. The topical formulation according to claim 1, wherein the olive oil is virgin olive oil extracted via a cold process method.

11. The topical formulation according to claim 1, wherein the formulation comprises from about 5% to about 10%, by weight of the total formulation of the olive oil.

12. The topical formulation according to claim 1, wherein the emulsifying ointment BP consists of:
Emulsifying Wax—30% (w/w);
White Soft Paraffin—50% (w/w); and
Liquid Paraffin—20% (w/w).

13. The topical formulation according to claim 1, wherein the formulation comprises a weight of emulsifying ointment BP to make up the final formulation concentration to 100% by weight.

14. The topical formulation according to claim 1, wherein the salicylic acid has the formula of Formula II

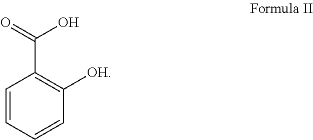

15. The topical formulation according to claim 1, wherein the formulation comprises a weight of salicylic acid of from about 2% to about 10% by weight of the total formulation.

16. The topical formulation according to claim 1, which is in the form of a cream or an ointment.

17.-19. (canceled)

20. A method of treating psoriasis or at least one symptom arising from psoriasis in a subject in need thereof, the method comprising topical application of the formulation according to claim 1 to an area of skin of the subject affected by psoriasis or at least one symptom arising from psoriasis until the psoriasis or at least one symptom arising therefrom is resolved.

21. The method according to claim 20, wherein the method comprises a first step of selecting a particular formulation having a concentration of the one or more Class 6 Low Potency corticosteroid(s) and optionally salicylic acid in the formulation appropriate for a particular phase of psoriasis or symptom(s) thereof of the subject and, wherein the particular phase is selected from one of:
an acute phase, including symptoms of itching, inflammation, scales, and/or rashes;
a scaly phase, including symptoms of thickening and/or discoloration of affected areas and including scale formation; and
a remission phase where there is no itching but including symptoms of mild scaling, dryness and/or inflammation of skin,
and the appropriate concentration of the one or more Class 6 Low Potency corticosteroid(s) and optionally salicylic acid is selected from one of:
where the subject is in the acute phase the formulation is an acute phase formulation having a concentration of the one or more Class 6 Low Potency corticosteroid(s) of about 0.0125% by weight of the total formulation and there is optionally between about 2% to about 10% of salicylic acid in the formulation;
where the subject is in the scaly phase, the formulation is a scaly phase formulation having a concentration of the one or more Class 6 Low Potency corticosteroid(s) of about 0.00625% by weight of the total formulation, and between about 2% to about 10% of salicylic acid by weight of the total formulation; and
where the subject is in the remission phase the formulation is a remission phase formulation having a concentration of the one or more Class 6 Low Potency corticosteroid(s) of about 0.0025% by weight of the total formulation, and there is no salicylic acid in the formulation.

22. The method according to claim 21, wherein where the subject is in the acute phase, the formulation is applied twice a day on affected areas of skin of the subject until resolution of acute phase symptoms of psoriasis, whereupon the subject enters the remission phase, followed by a phased discontinuation of use of the acute phase formulation comprising a once-daily application for about 5 days, followed by a once-daily application on alternate days for the next about 10 days, and finally, treatment of the subject with the remission phase formulation as required by the subject.

23. The method according to claim 21, wherein where the subject is in the scaly phase, the formulation is applied twice a day on affected areas of skin of the subject until resolution of scaly phase symptoms of psoriasis, whereupon the subject enters the remission phase, followed by a phased discontinuation of use of the scaly phase formulation comprising a once-daily application for about 5 days, followed by a once-daily application on alternate days for the next about 10 days, and finally, treatment of the subject with the remission phase formulation as required by the subject.

24. The method according to claim 21, wherein where the subject is in the remission phase, the remission phase formulation is applied as required.

25. The method according to claim 21, wherein the remission phase formulation is applied about twice-daily by the subject to the affected skin requiring treatment.

26. A method of producing a topical formulation according to claim 1, comprising mixing the one or more Class 6 Low Potency corticosteroid(s), the emulsifying ointment BP, the honey, the olive oil, and optionally salicylic acid until combined.

27.-30. (canceled)
